(19) United States
(12) Patent Application Publication
(10) Pub. No.: US 2023/0134629 A1
(43) Pub. Date: May 4, 2023

(54) ON-BONE ROBOTIC SYSTEM FOR COMPUTER-ASSISTED SURGERY

(71) Applicant: ORTHOSOFT ULC, Montreal (CA)

(72) Inventors: Louis-Philippe AMIOT, Montreal (CA); Pierre COUTURE, Montreal (CA); Karine DUVAL, Montreal (CA); Alain RICHARD, Lachine (CA)

(21) Appl. No.: 18/052,024

(22) Filed: Nov. 2, 2022

Related U.S. Application Data
(60) Provisional application No. 63/274,554, filed on Nov. 2, 2021.

(51) Int. Cl. A61B 17/68; A61B 34/20

(52) U.S. Cl. CPC A61B 17/686; A61B 34/20; A61B 2034/2057; A61B 2034/2059

(57) ABSTRACT

An on-bone robotic system may have a bone anchor device configured to be received in a bone, the bone anchor device including at least one sensor for tracking an orientation of the bone. A robotic tool unit may be releasably connected to the bone anchor device, the robotic tool unit including one or more actuators for displacing a surgical implement of the robotic tool unit relative to the bone when the robotic tool unit is connected to the bone anchor device. The on-bone robotic system includes one or more joints enabling a degree(s) of freedom of movement of the surgical implement relative to the bone anchor device. The on-bone robotic system includes a processor for operating the at least one actuator as a function of the tracking of the bone by the sensor.

### CROSS-REFERENCE TO RELATED APPLICATION

The present application claims the priority of U.S. Patent Application No. 63/274,554, filed on Nov. 2, 2021 and incorporated herein by reference.

### TECHNICAL FIELD

The application relates to computer-assisted surgery and, more particularly, to robotic tools, roboticized tools and implantable electronics used in surgical procedures.

### BACKGROUND

In orthopedic surgery, robots are increasingly used to perform bone resection, to guide the positioning of implants, among other actions, in the context of computer-assisted surgery. Whether the robots are of collaborative nature or autonomous, the use of robots may contribute to increasing the precision and accuracy of bone-altering procedures. Robotic arms are tracked so as to navigate their various implements relative to the bone, i.e., obtain position and/or orientation data relating the robot implements to bone landmarks.

However, robots tend to have a non-negligible footprint in the operating room. Robotic systems typically have their own stand and/or station, and may consequently be an obstacle limiting personnel movement around the patient. Moreover, in some instances, robotic systems are used jointly with voluminous tracking systems, such as optical tracking devices, that also add to the space management concern in the operating room. It would be desirable to reduce the footprint of robots used in surgical procedures.

### SUMMARY

In a first aspect, there is provided an on-bone robotic system comprising a bone anchor device configured to be received in a bone, the bone anchor device including at least one sensor for tracking an orientation of the bone; a robotic tool unit releasably connected to the bone anchor device, the robotic tool unit including at least one actuator for displacing a surgical implement of the robotic tool unit relative to the bone when the robotic tool unit is connected to the bone anchor device; wherein the on-bone robotic system includes at least one joint enabling at least one degree of freedom of movement of the surgical implement relative to the bone anchor device; and wherein the on-bone robotic system includes a processor for operating the at least one actuator as a function of the tracking of the bone by the sensor.

Further in accordance with the first aspect, for example, the bone anchor device has a receptacle configured to be received in the bone, the receptacle accommodating the at least one sensor.

Still further in accordance with the first aspect, for example, a leading end of the bone anchor device is flared.

Still further in accordance with the first aspect, for example, an anti-rotation feature projects laterally from the receptacly.

Still further in accordance with the first aspect, for example, the anti-rotation feature includes at least one fin.

Still further in accordance with the first aspect, for example, the at least one sensor includes an inertial sensor.

Still further in accordance with the first aspect, for example, the bone anchor device includes a battery.

Still further in accordance with the first aspect, for example, the bone anchor device is configured to be used as an implant to track movement of the bone post-operatively.

Still further in accordance with the first aspect, for example, the at least one actuator includes at least one motor.

Still further in accordance with the first aspect, for example, there may be two of the motor, the robotic tool unit displacing the surgical implement in at least two rotational degrees of freedom.

Still further in accordance with the first aspect, for example, the at least one actuator includes at least one linear actuator.

Still further in accordance with the first aspect, for example, the surgical implement has a cut slot.

Still further in accordance with the first aspect, for example, the robotic tool unit includes at least one sensor for tracking an orientation of the surgical implement.

Still further in accordance with the first aspect, for example, the robotic tool unit includes at least one camera oriented toward the bone and configured to capture images of the bone.

Still further in accordance with the first aspect, for example, a communication device may be connected to the processor and configured for wireless communication.

In accordance with a second aspect of the present disclosure, there is provided a method for performing an orthopedic procedure comprising: anchoring an on-bone robotic system to a bone via a bone anchor device inserted in the bone, the bone anchor device including at least one sensor for tracking an orientation of the bone; operating the on-bone robotic system for the on-bone robotic system to displace a surgical implement operatively connected to the bone anchor device, a movement of the surgical implement being guided as a function of the tracking of the bone by the sensor; and detaching at least the surgical implement from the bone anchor device to leave the bone anchor device as an implant post-operatively, the bone anchor device configured to track the bone post-operatively.

Further in accordance with the second aspect, for example, anchoring the on-bone robotic system to the bone including drilling a hole in the bone for insertion of the bone anchor device in the hole.

Still further in accordance with the second aspect, for example, insertion of the bone anchor device in the hole includes having an anti-rotation feature penetrate the bone.

Still further in accordance with the second aspect, for example, the movement in the operating includes moving the surgical implement in at least one rotational degree of freedom.

Still further in accordance with the second aspect, for example, moving the surgical implement includes actuating a rotational motor to move the surgical implement in the at least one rotational degree of freedom.

Still further in accordance with the second aspect, for example, the movement in the operating includes moving the surgical implement in two rotational degrees of freedom.

Still further in accordance with the second aspect, for example, the movement in the operating includes moving the surgical implement in one translational degree of freedom.

Still further in accordance with the second aspect, for example, the method may include imaging the bone from the on-bone robotic system.

Still further in accordance with the second aspect, for example, the method may include matching the imaging of the bone from the on-bone robotic system with a pre-operative virtual model of the bone for navigating a position and orientation of the surgical implement relative to the bone.

Still further in accordance with the second aspect, for example, the method may include wirelessly communicating data from the at least one sensor.

In accordance with a third aspect, there is provided a system for tracking a bone intraoperatively in a surgical procedure and post-operatively, comprising: a processing unit; and a non-transitory computer-readable memory communicatively coupled to the processing unit and comprising computer-readable program instructions executable by the processing unit for: obtaining orientation data of at least one sensor in a bone anchor device anchored to a bone, intraoperatively; actuating at least one actuator to displace a surgical implement operatively connected to the bone anchor device as a part of an on-bone robot, as a function of the orientation data; and after the surgical procedure, obtaining orientation data of at least one sensor in the bone anchor device remaining anchored to the bone, post-operatively.

Further in accordance with the third aspect, for example, actuating at least one actuator includes actuating at least one rotational motor to orient the surgical instrument relative to the bone in one rotational degree of freedom.

Still further in accordance with the third aspect, for example, actuating at least one actuator includes actuating a second rotational motor to orient the surgical instrument relative to the bone in a second rotational degree of freedom.

Still further in accordance with the third aspect, for example, actuating at least one actuator includes actuating at least one linear actuator to displace the surgical instrument relative to the bone in a translational degree of freedom.

Still further in accordance with the third aspect, for example, the method may include imaging the bone from the on-bone robot.

Still further in accordance with the third aspect, for example, the method may include matching the imaging of the bone from the on-bone robot with a pre-operative virtual model of the bone for navigating a position and orientation of the surgical implement relative to the bone.

### DESCRIPTION OF THE DRAWINGS

Reference is now made to the accompanying figures in which:

FIG. 1 is a schematic view of an on-bone robotic system in accordance with an aspect of the present disclosure;

FIGS. 2A and 2B are schematic views showing the on-bone robotic system of FIG. 1 relative to a distal femur;

FIGS. 3A, 3B and 3C are schematic views of the on-bone robotic system of FIG. 1, with an alignment plate implement;

FIGS. 4A and 4B are a schematic views of the alignment plate implement with bone contacting actuators in accordance with an aspect of the present disclosure;

FIGS. 5A to 5D are schematic illustrations of the robotic system of FIG. 1 with a cutting guide implement;

FIGS. 6A to 6C are a series of views showing the on-bone robotic system of FIG. 1, as used on a tibia in accordance with an aspect;

FIGS. 7A to 7C are a series of views showing the on-bone robotic system of FIG. 1, as used on a tibia in accordance with another aspect;

FIGS. 8A to 8E are schematic views of the on-bone robotic system of FIG. 1 using a provisional implant surgical implement;

FIG. 9 is a perspective view of a variant of a cutting block surgical implement of the on-bone robotic system of FIG. 1;

FIG. 10 is a schematic perspective view of another variant of a cutting block surgical implement of the on-bone robotic system of FIG. 1; and

FIG. 11 is a schematic side view of another variant of a cutting block surgical implement of the on-bone robotic system of FIG. 1.

### DETAILED DESCRIPTION

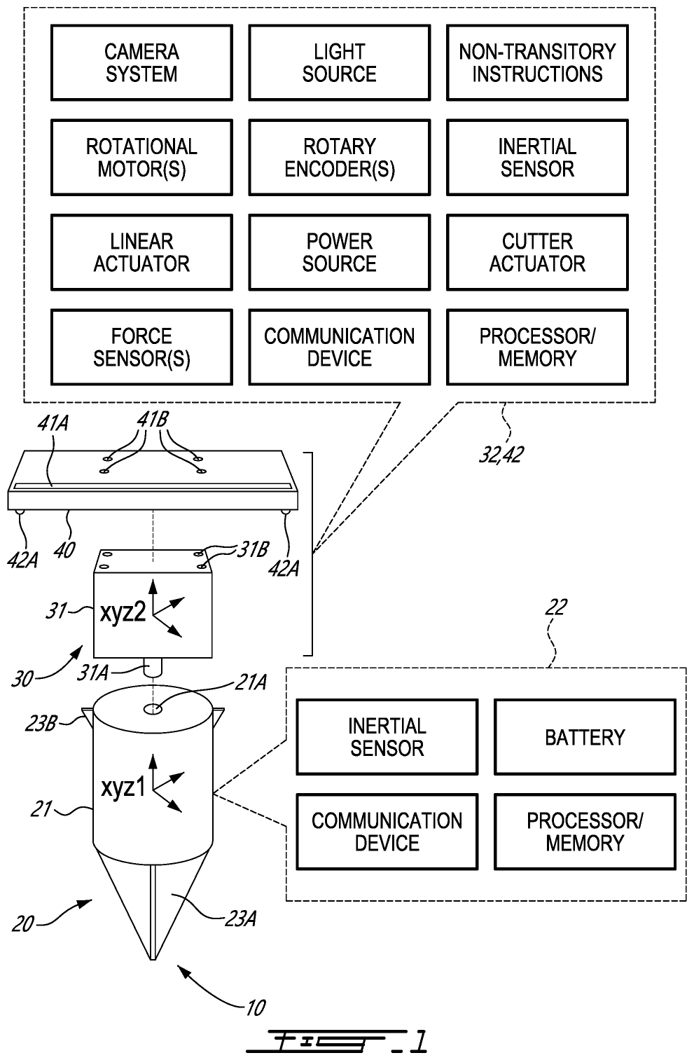

Referring to the drawings and more particularly to FIG. 1, there is illustrated an on-bone robotic system at 10. The on-bone robotic system 10 is of the type used as part of computer-assisted surgery, to provide guidance to an operator in performing orthopedic surgery. Accordingly, the on-bone robotic system 10 may have electronic components and actuators so as to perform some automated functions described herein, and/or to guide an operator in performing alterations to a bone and placing implants (onboard electronics). Moreover, the on-bone robotic system 10 may include components that may be implanted in the patient's body (occasionally referred to as a wearable), that can provide navigation data intra-operatively and optionally post-operatively. In the following figures, the robotic system 10 is shown in a knee replacement surgical procedure that involves the resection of bone to define cut planes on a distal femur and at a tibial plateau. However, it is contemplated to use the on-bone robotic system 10 for other types of surgical procedures.

In FIG. 1, the on-bone robotic system 10 is shown in a schematic manner, as having a bone anchor device 20 and a robotic tool unit connectable to the bone anchor device 20. The robotic tool unit may include a robotic base 30 and an exemplary surgical implement 40, that may be integrally connected or releasably connected to one another. Other surgical implements that may be part of the robotic tool unit are shown as 50, 60, 70 and 80 and are described hereinbelow. The robotic base 30 and the surgical implement 40 are shown as being separated and interconnectable, but they may be as one component that may be connected to the bone anchor device 20. Herein, for simplicity, components of the bone anchor device 20 will be in the 20s, such as receptacle 21, etc. The same nomenclature is used for the robotic base 30 and for the surgical implements 40, 50, 60, 70 and 80. The bone anchor device 20 may perform different functions. It may serve as an anchor or attachment for other components of the on-bone robotic system 10. It may also be configured to track the bone to which it is connected, such as by providing orientation data related to the bone. For example, the bone anchor device 20 may produce data indicative of a location of a mechanical axis of a bone. The bone anchor device 20 may also be used as an implanted electronic device, to provide bone related data post-operatively, such as movements associated with a gait, e.g., range of motion, with flexion/extension, forces, step count, stride length, among others. The robotic tool unit attaches to the bone anchor device 20 with its robotic base 30 and is used intra-operatively to perform various functions associated for example with the surgical implement(s) 40 connected to the robotic base 30. The robotic base 30 may be separated from the bone anchor device 20, for embodiments in which the bone anchor device 20 becomes a post-operative implanted electronic device.

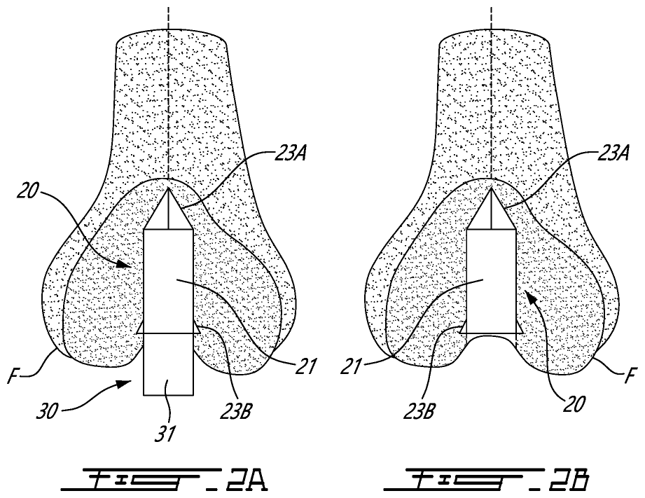

Referring concurrently to FIGS. 1, 2A and 2B, the bone anchor device 20 is of the type that penetrates into a bone. In the embodiment of FIGS. 2A and 2B, the bone anchor device 20 is anchored to a distal femur F, and may be used to track bone landmarks of the femur F, such as a mechanical axis, in three-dimensional space. Values such as varus/valgus and flexion/extension may be derived from the mechanical axis, whereby the tracking of the mechanical axis via the bone anchor device 20 may serve this purpose.

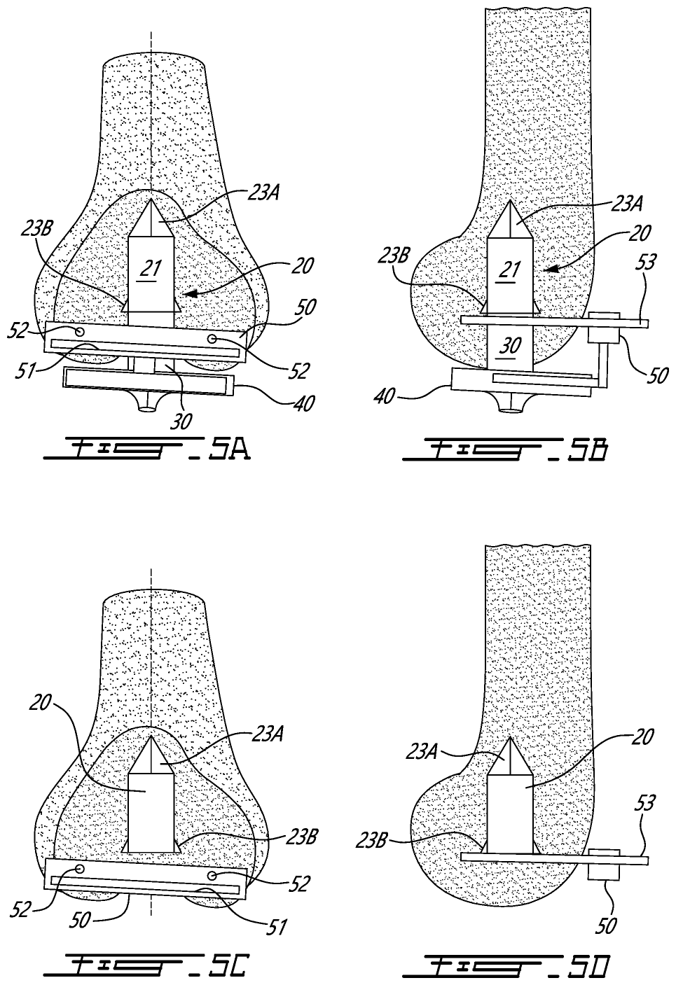

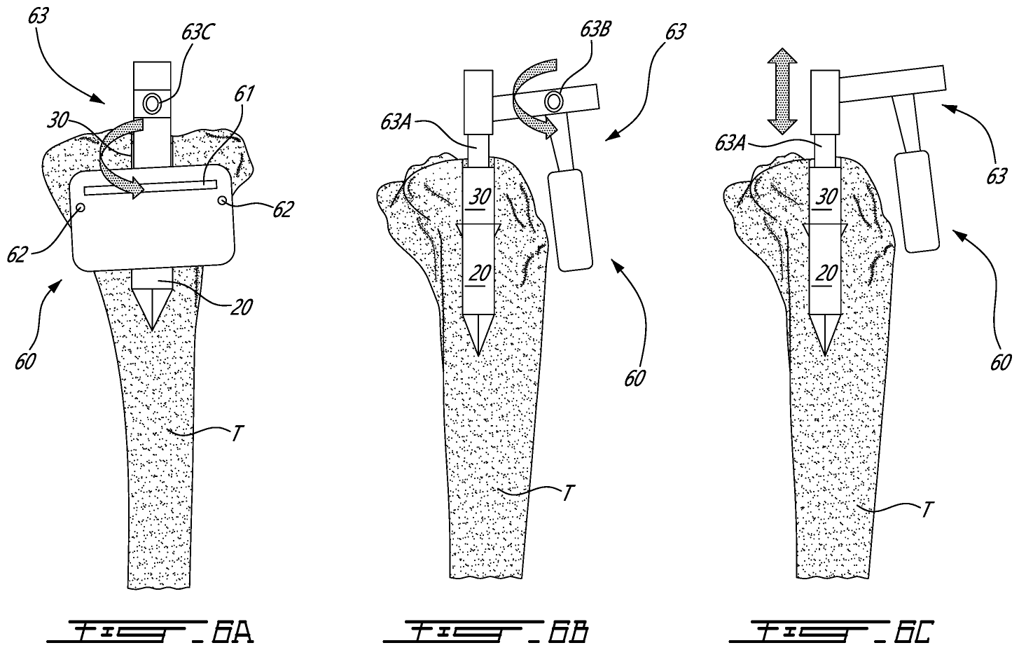

The bone anchor device 20 is configured to be received in a cavity in the bone. For example, as shown in FIGS. 2A and 2B, the bone anchor device 20 is received in a cavity formed in the intercondylar fossa of the distal femur F, as one possible location for receiving the bone anchor device 20. FIGS. 6A-6C and 7A-7C show the bone anchor device 20 in the proximal tibia. The bone anchor device 20 encloses electronic components and therefore defines a receptacle 21 or like body to accommodate the electronic components. The receptacle 21 in FIG. 1 is schematically shown as being cylindrical in shape, but may have other shapes. In an embodiment, it is considered to drill a hole in the bone so as to introduce therein the bone anchor device 20, with the cylindrical shape of the receptacle 21 being well suited to be received in a drilled hole. The receptacle 21 is configured to be connected to the robotic base 30 and therefore may have a connector 21A. In the illustrated embodiment, the connector 21A is shown as being a hole (e.g., threaded hole), but may have other forms, such as projecting members like a shaft, a rod, or may integrate a quick-connect system features, etc. The connector 21A is complementary to a connector of the robotic base 30 and is selected for the connection between the bone anchor device 20 and the robotic base 30 to be geometrically determined, i.e., once the bone anchor device 20 and the robotic base 30 are connected to one another, some geometrical data is known, such as a distance between the bone anchor device 20 and the robotic base 30, an orientation between coordinate system xyz1 and xyz2 associated respectively with the bone anchor device 20 and the robotic base 30, if movement between the bone anchor device 20 and the robotic base 30 is possible after interconnection. Indeed, the connector 21A may be part of a joint allowing relative movement between the bone anchor device 20 and the robotic base 30. The joint(s) may include a spherical joint, a universal joint, and a telescopic joint, for example.

Electronic components 22 are received in the receptacle 21 of the bone anchor device 20. In an embodiment, the bone anchor device 20 is autonomous in that it may operate in and of itself to produce signals. Therefore, as part of the electronic components 22, there may be a processor/memory to execute particular functions. The memory may include non-transitory instructions executable by the processor to perform given functions detailed below. As the bone anchor device 20 may remain implanted in the bone post-surgery, a power source such as a battery may be part of the electronic components 22. The bone anchor device 20 as set out above is tasked with tracking the bone in space. Therefore, an inertial sensor(s) is part of the electronic components. The inertial sensor may be known as a sourceless sensor, a micro-electromechanical sensor unit (MEMS unit), and has any appropriate set of inertial sensors (e.g., accelerometers, gyroscope) to produce tracking data in at least three degrees of rotation (i.e., the orientation about a set of three axes is tracked). The inertial sensor may include a processor, including a printed circuit board, and a non-transitory computer-readable memory communicatively coupled to the processor and comprising computer-readable program instructions executable by the processor, or may use the processor/memory described above. Moreover, the inertial sensor may be self-contained, in that they may be pre-calibrated for operation, have their own powering or may be connected to a power source, and may have an interface, such as in the form of a display thereon (e.g., LED indicators).

Further, as part of the electronic components, a communication device may be present for the bone anchor device 20 to issue signals indicative of the orientation of the bone. The communication device may be a wireless device that may use any appropriate wireless communication protocol, such as Bluetooth®, Wi-Fi, etc.

It is desired that the bone anchor device 20 remain anchored in a fixed position and orientation relative to the bone. In a variant, it may be possible to impact the bone anchor device 20 in the bone. Therefore, a spike 23A or like flaring end (e.g., frusto-conical end) may project from a leading end of the bone anchor device 20, as projecting from the receptacle 21, the flaring shape being from the tip toward the trailing end. The spike 23A is shown as having triangular fins that may facilitate the impacting of the bone anchor device 20 into the bone. However, if the bone anchor device is received in a drilled hole in the bone, the spike 23A may be optional. Moreover, considering the penetration of the bone anchor device 20 into the bone, the spike 23A may be received in cancellous bone, which may or may not provide sufficient purchase. Accordingly, one or more fins 23B or like anchoring features may be at or near a trailing end of the receptacle 21, for the fins 23B to purchase into cortical bone. The fins 23B may have a smaller profile than the spike 23A, that may suffice in preventing rotation of the receptacle 21 in the bone, and ensure that the bone anchor device 20 does not move relative to the bone. Other anti-rotation features may be present as well. The fins 23B may have a flaring profile from a leading to trailing direction to facilitate interaction with the surrounding bone.

For the inertial sensor within the electronic components 22 to perform a tracking of the axis of the bone receiving the bone anchor device 20, appropriate calibration techniques may be used. In a variant, calibration is performed to create the axes or other landmarks. For example, the mechanical axis may be determined using the method described in U.S. Pat. No. 9,901,405, incorporated herein by reference. Other data that may be tracked by the bone anchor device 20 may include other axes, such as the medio-lateral axis of the femur, the frontal plane of the femur, a bone model of the femur, etc, in the context of the femur. In terms of pre-calibration, the position and orientation of the inertial sensor within the receptacle 21 may be known such that the inertial sensor may be associated to a given landmark of a bone upon insertion. For example, the bone anchor device may be calibrated relative to the entry point of a mechanical axis (e.g., tibia) by the its positioning in a drilled hole at the entry point in the tibia.

In order to accommodate the electronic components 22, and to limit its invasiveness, the receptacle 21 has a given volumetric size. In an embodiment, a diameter of the receptacle 21 is between 8 mm and 10 mm, though other dimensions may be possible. A height of the receptacle 21 may be between 8 and 15 mm, though it may be smaller or larger than that.

Referring to FIG. 1, the robotic base 30 and the surgical implement 40 may form part of the robotic tool unit that is used with the bone anchor device 20, to perform given tasks on the bone. The robotic tool unit may be available as a whole, i.e. integrating the robotic base 30 and the surgical implement 40 together, though it may be constituted of detachable components. i.e., the robotic base 30 and the surgical implement 40 being releasably connected. The releasable connection may allow the use of different surgical implements 40 with a same robotic base 30, thereby reducing the cost of the robotic tool units as a common robotic base 30 with its electronic and mechanical components may be shared by the surgical implements 40. During a surgical procedure, the robotic tool unit is moved relative to the bone and may be used by the user as a physical interface to perform functions on the bone, while the bone anchor device 20 is anchored to the bone and serves as a base for the robotic tool unit.

In FIG. 1, the robotic base 30 is in an exploded relation with the bone anchor device 20. The robotic base 30 may be releasably connectable to the bone anchor device 20. The robotic base 30 may also define a receptacle 31 so as to receive therein electronic, mechanical and/or electro-mechanical components 32,42. The electronic, mechanical and/or electro-mechanical components 32,42 may also be within the surgical implement 40, hence the use of reference numeral 42. Stated differently, the electronic and/or mechanical components 32,42 may be part of the robotic tool unit, i.e., a combination of the robotic base 30 and the surgical implement 40. The receptacle 31 has a connector 31A that is configured to be connected to the connector 21A of the bone anchor device 20. For example, the connector 31A is shown as being a shaft, as one possible means to be connected to the connector 21A of the bone anchor device 20. In an embodiment, the connectors 21A and 31A concurrently define one or more joints, to allow given movements of the robotic tool unit relative to the bone anchor device 20. For example, with reference to xyz1 in FIG. 1, i.e., the referential system of the bone anchor device 20 that is fixed to the bone, the robotic tool unit, including the robotic base 30 and/or the surgical implement 40, may move in translation toward and away from the bone anchor device 20, e.g., in a direction generally parallel to the mechanical axis of the femur F. The movement in translation may be limited to one degree of freedom (DOF). The robotic tool unit, including the robotic base 30 and/or the surgical implement 40, may also rotate relative to the bone anchor device 20, in two or three DOFs. One rotational DOF of a joint between the robotic tool unit and the bone anchor device 20 may be aligned with the femur for rotation about a medio-lateral axis of the femur F, for flexion-extension plane adjustment. Another rotational DOF of the joint between the robotic tool unit and the bone anchor device 20 may be aligned with the femur for rotation about an anterior-posterior axis of the femur F, for varus-valgus adjustment. A third rotational DOF may be aligned with the axis of the bone anchor device 20, allowing a rotational adjustment about the posterior condyles or the epicondyles of the femur. This may allow an adjustment using the condyle abutment member described below.

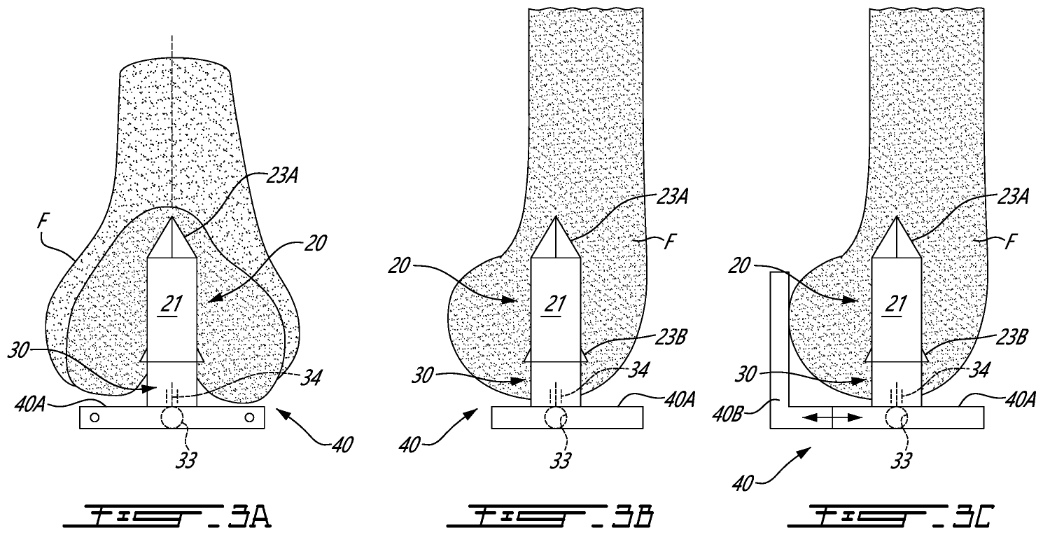

Connectors 31B may also be provided on the receptacle 31 for connection of the surgical implement(s) 40 to the robotic base 30, if they are not integrally connected. The connectors 31B are shown as being threaded holes, but other connection components may be present, for instance quick connect features such as clips, tongues, etc, or other types of complementary connections. In a variant, the robotic base 30 is fixed in movement relative to the bone anchor device 20, while the surgical implement(s) 40 may move relative to the robotic base 30 and thus relative to the bone anchor device 20, by one or more joints between the robotic base 30 and the surgical implement 40. The robotic base 30 and the surgical implement 40 may be releasably connected, as shown in FIG. 1, with connector holes 41B aligned with the holes 31B in the robotic base 30, for fastener connection, as a possibility. The movements may be as described above for a joint between the bone anchor device 20 and the robotic base 30, i.e., one translational DOF, and two or more rotational DOFs. FIGS. 3A, 3B and 3C show an exemplary spherical joint 33 and a translational joint 34 between the surgical implement 40 and the robotic base 30, to illustrate one contemplated manner to move the surgical implement 40 relative to the femur F, in two or more rotational degrees of freedom. Other joint arrangements are possible to provide any suitable or desired degrees of freedom of movement. As an example, the surgical implement 40 has a cut slot 41A, but may have different and/or other guiding features (e.g., drill guides, abutment features, etc).

Among the electronic and/or mechanical components 32,42, the robotic base 30 may include a processor/memory having non-transitory instructions for the processor to perform given functions associated with the surgery. Rotational motors may be provided in the electronic and/or mechanical components 32,42 and may be used to control rotation of the robotic base 30 relative to the bone anchor device 20 or of the robotic base 30 relative to the surgical implement 40. Movements of the robotic base 30 may be also be controlled using microgears, linear actuators or fluids (air, oil, water). An example thereof is provided below. In an embodiment, the rotational motors are controllable to cause movement of the receptacle 31 relative to the connector 31A, with the connector 31A being part of the joint between the bone anchor device 20 and the robotic base 30. Therefore, with the surgical implement 40 connected to the robotic base 30, movement of the robotic base 30 may cause movement of the surgical implement 40 relative to the bone. A linear actuator may be present as part of the components 32,42 and may actuate the translational movement between the robotic base 30 and the bone anchor device 20. Stated differently, the robotic base 30 may move closer or farther from the bone anchor device 20. Force sensors may also be present as part of the components 32,42 in the robotic base 30 or may be in the surgical implement 40. Rotary encoders may be present to determine an orientation of the robotic base 30 relative to the bone anchor device 20 if one is moveable relative to the other by way of one or more joints. Alternatively, the rotary encoders may determine an orientation of the surgical implement 40 relative to the robotic base 30 if one may rotate relative to the other. Any appropriate power source is part of the components 32,42. For example, the robotic tool unit may be wired to a power source, or may have a battery. A communication device may also be present for communication between the robotic tool unit and the bone anchor device 20 or with a processor separate from the on-bone robotic system 10. While rotary encoders may determine the relative orientation between the robotic base 30 and the bone anchor device 20, an inertial sensor may be present in the robotic base 30 or the surgical implement 40 to monitor an orientation of the robotic tool unit. It is also possible to use optical tracking technologies to observe a rotation of the robotic base 30 relative to the bone and/or bone anchor device. For example, the optical tracking technologies may include laser rangefinders that are part of the robotic base 30 and that project light, for instance on the bone. One or more cameras may also be provided as part of the components 32,42, the expression “camera” encompassing the various hardware and software components necessary to perform imaging (e.g., lens(es), aperture, image sensor such as CCD, image processor). The cameras may come as a set to operate as a depth camera system. The cameras may be on the robotic base 30 and/or on the surgical implement 40, with suitable distance given to the lenses of the camera(s) to observe the bone to which the on-bone robotic system 10 is mounted and/or to observe the environment of the bone—lenses shown at 42A in FIG. 1 being an example. For instance, the camera(s) may be used to image a bone surface. The imaging may then be used to match the imaged bone surface to a bone model (e.g., 3D virtual bone model) obtained via different preoperative or intraoperative imaging (e.g., CT scans, radiography in its various forms), and programmed into the memory of the on-bone robotic system 10 or accessible by the on-bone robotic system 10. Hence, the presence of camera(s) 32,42, on the on-bone robotic system 10 may contribute to the calibrating of the system relative to the bone, and to the subsequent navigation. The camera(s) 32,42 may for example have a unique perspective of voids, depressions on bones. As another possibility, a cutter actuator may be present as part of the robotic tool unit, if the surgical implement 40 is configured to perform cuts, as described hereinbelow. The cutter actuator may be a motor(s), an ultrasonic oscillator, a linear actuator, etc.

Now that the general configuration of the on-bone robotic system 10 has been described, a surgical procedure involving the system 10 is set forth, by which different types of surgical implements 40 may be used. The surgical procedure is a knee replacement procedure, in which a tibial plateau implant is installed on a tibia, and a femoral component is implanted on the distal femur. The on-bone robotic system 10 may be used in other types of surgery, for instance with a partial proximial tibia procedure, distal femur only, proximal tibia only, hip surgery (e.g., partial hip replacement, total hip replacement), hip resurfacing, shoulder surgery, etc.

As a starting point, the bone anchor device 20 is installed in the bone. For example, the bone anchor device 20 is in the intercondylar fossa (e.g., within the intramedullary canal, or medullar canal), and is tasked with tracking a landmark of the femur F, such as a referential system including a mechanical axis. Other locations on the femur F are also possible for the bone anchor device 20.

Referring to FIGS. 3A, 3B and 3C, anterior and side views of the femur with the on-bone robotic system 10 are provided. The surgical implement 40 is shown as being an alignment plate that may be displaceable so as to contact the distal aspects of the condyles. Accordingly, the alignment plate has an abutment plane 40A, and joint(s) in the robotic tool unit or between the robotic tool unit and the bone anchor device 20 may allow the abutment plane 40A to be brought into contact with the condyles, by translation and/or rotation. Though FIGS. 3A and 3B show a single plane for abutment with the distal aspects of the condyles, the surgical implement 40 may also have another abutment plane for abutment with the posterior aspects of the condyles, such as shown in FIG. 3B. In FIG. 3B, a condyle abutment member 40B may be connected to the abutment plane 40A, though alternatively it may be possible to have the condyle abutment member 40B integrally part of the abutment plane 40A. A translation movement between the abutment plane 40A and the condyle abutment member 40B is possible in an embodiment, by way of a translation joint. In an embodiment, the bone contact surfaces of the abutment plane 40A and of the condyle abutment member 40B are perpendicular relative to one another. The abutment contact may be automated by the on-bone robotic system 10, with the force sensors determining if contact is achieved. With the components 22 and 32,42, the orientation of the surgical implement 40 relative to the bone anchor device 20 may be known by sharing of orientation data, such that additional bone landmarks may be tracked. In a variant, the alignment plate is used to locate the medio-lateral axis, a plane of the posterior aspects of the condyles, and/or a plane of the distal aspects of the condyles and/or a plane aligned with both epicondyles. Once the mechanical axis is known, the robotic base 30 can align itself parallel to the mechanical axis and, using the actuation means described herein, may touch the most distal part of the condyle with the abutment plane 40 and record that landmark (most distal point of the femur), Bone cuts can then be made relative to that landmark, e.g., resect a plane 9 mm from the most distal femoral point. Also, the orientation of the cutting plane for the distal cut may include the palpation of the distal condyles with an angle of flexion, e.g., 3°, relative to the mechanical axis.

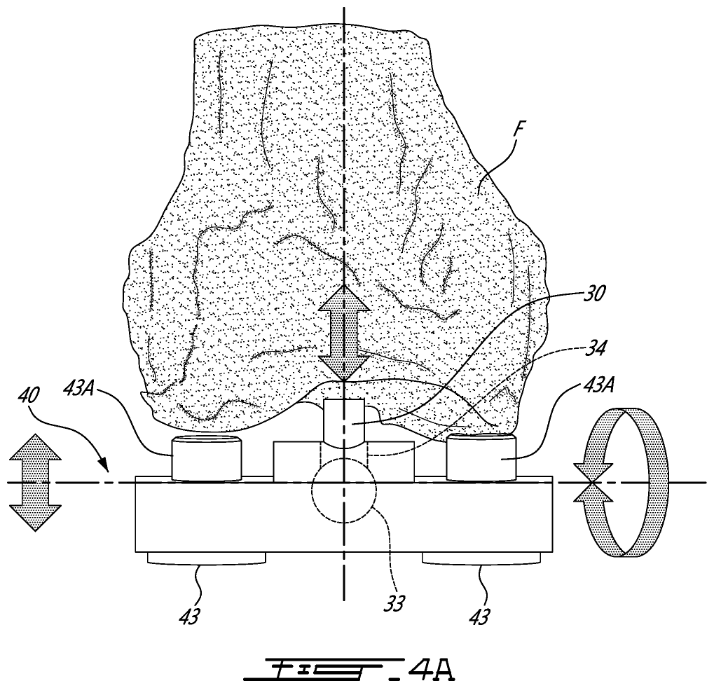

Referring to FIG. 4A, a variant of the abutment plate surgical implement 40 is illustrated, in which bone-contacting actuators 43 are provided at the corners or sides of the abutment plane 40A. The bone-contacting actuators 43 each have a piston or like movable component 43A projecting out of the abutment plane 40A. The movable components 43A are configured to contact given landmarks of the bone, such as the distal features of the condyles. For example, the bone-contacting actuators 43 are stepper motors, ball-screw motors, or equivalents, that have an output rod defining the movable components 43A. Rotation of the bone-contacting actuators 43, may result in a projecting movement of the movable components 43A, and may hence be performed for adjusting the orientation of the surgical implement 40 relative to the bone for instance via the spherical joint 33. Concurrent rotation of the bone-contacting actuators 43 may also be performed to cause a spacing of the abutment plane 40A from the bone, via the translational joint 34.

For example, there may be four such bone-contacting actuators 43, though only two are visible from the point of view of FIG. 4A. Therefore, as the abutment plate surgical implement 40 may have its orientation known relative to the bone axis via the bone anchor device 20 (e.g., inertial sensor in the electronic components 22), the bone-contacting actuators 43 may be controlled to orient the abutment plate surgical implement 40 to a desired orientation, relative to anatomical features of the femur, such as the mechanical axis, and/or to space the abutment plate surgical implement 40 from the femur F. It is therefore possible to allow a varus/valgus adjustment and/or flexion/extension slope adjustment of an eventual resection plane via the orientation of the surgical implement 40 relative to the femur F, notably by the degrees of freedom present in the robotic tool unit, or between the bone anchor device 20 and the robotic base 30. The control of the bone-contacting actuators 43 may be used to set the abutment plate surgical implement 40 to a desired orientation and/or position, and hold the abutment plate surgical implement 40 in the desired orientation. If the bone-contacting actuators 43 are operated concurrently, it is also possible to move the surgical implement 40 axially relative to the bone, if a translational degree of freedom is present in the robotic tool unit or between the bone anchor device 20 and the robotic base 30. The bone-contacting actuators 43 may be self-locking in that they may hold their length unless actuated. Therefore, once the bone-contacting actuators 43 hold their length and abut the bone, the abutment plate surgical implement 40 may be in a fixed position and orientation relative to the bone, for example as hovering over the bone, and can serve as a structure to support additional components. The desired position and/or orientation may be automated and/or effect on-bone, with the robotic system 10 operated to achieve the desired position and/or orientation for the abutment plate surgical implement 40.

Referring to FIG. 4B, another embodiment is shown, in which the abutment plate surgical implement 40 has the movable components 43A displaceable using cylinders, also known as pistons, shown as 43B, whether there are two or more of the cylinders 43B. The cylinders 43B may be hydraulic or air powered cylinders, etc. As described in U.S. Patent Application Publication 2009/0018544A1 to Zimmer, Inc., which is incorporated herein by reference , each cylinder may have its own valve to control the length of the cylinder 43B. The pressure source may be integrated, or may be separate from the on-bone robotic system 10.

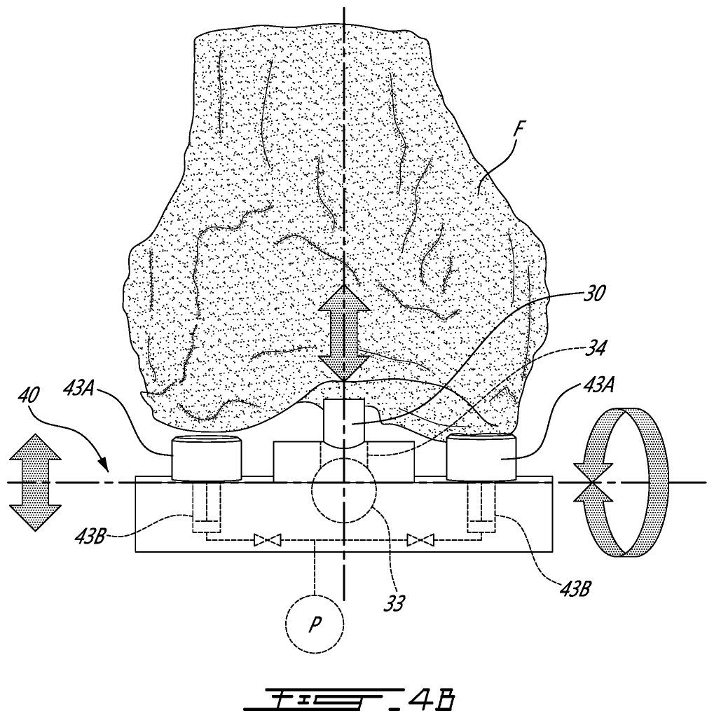

Referring to FIGS. 5A and 5B, once the desired position and/or orientation is achieved for the abutment plate surgical implement 40 relative to the femur, another implement, such as a cutting guide 50, may be secured to the abutment plate surgical implement 40. The cutting guide 50 may have one or more cut slot(s) 51 and pinholes 52 for the cutting guide 50 to be secured to the bone. The exemplary embodiment is configured for the creation of a distal cut, but other cut slots may be present, for other cuts such as the anterior cut, the anterior chamfer, the posterior chamfer, and/or the posterior cut. The cut generated using the cut slot 51 may be a provisional cut, for instance to support a provisional implant.

The cutting guide 50 is in a known geometrical relation with respect to the abutment plate surgical implement 40 when attached to it, such that a cut plane machined via the cut slot 51 is in a desired position and orientation relative to the bone. The on-bone robotic system 10 may be operated to guide in the resection of cut planes in a navigated orientation relative to bone landmarks tracked by the bone anchor device 20, such as the mechanical axis of the femur F, taking into consideration the geometry of the cutting guide 50 and the geometrical relation between the cutting guide 50 and the surgical implement 40 when displacing the surgical implement 40. Therefore, following FIG. 4A or FIG. 4B in which an orientation of the abutment plate of the surgical implement 40 is adjusted via the electronic components 22 and 32, and FIGS. 5A and 5B in which the cutting guide 50 is secured to the surgical implement 40 to having the cut slot(s) 51 at a desired location, the cutting guide 50 may be pinned to the bone, with pins 53 as in FIG. 5B, or attached to it in another other manner. The cameras 32 may be used to provide video imaging by which the cutting guide 50 may be positioned and oriented relative to the bone. The robotic tool unit (i.e., including the robotic base 30 and the surgical implement 40), may be removed to enable the distal cut. The bone anchor device 20 may remain in the bone after the cutting guide 50 is secured to the bone, and be used to track movements of the bone as described above.

Consequently, the on-bone robotic system 10 featuring the surgical implements 40 and/or 50 (the cutting guide 50 and the alignment plate surgical implement 40 may be a single device) may self-align relative to the femur F, by performing its femoral registration, and may guide femoral cuts. The self-alignment may also involve the imaging using the cameras 32, for example using a 3D model of the bone. Moreover, the imaging from cameras or laser(s) from the components 32,42 may be used to determine the depth of resection relative to a landmark (e.g., malleoli for the tibia), such that laxity values can be calculated using virtual implant geometries. If the bone anchor device 20 is a implanted electronic device that is used post-operatively, the coordinates of the various planes resulting from the femoral registration may be transferred to the electronic components 22 of the bone anchor device 20, as data used in the post-operative tracking.

Referring now to FIGS. 6A to 6C, the on-bone robotic system 10 may also be used to create a cut plane on the proximal tibia T, to define a tibial plateau for receiving an implant. Accordingly, the on-bone robotic system 10 may have the bone anchor device 20, and the robotic tool unit including the robotic base 30 and surgical implements of different types. In FIGS. 6A to 6C, the cutting implement is defined by a cutting guide 60 having a cut slot 61, and pinholes 62 for securing the cutting guide 60 to the tibia T. The pinholes 62 are one solution among others to secure the cutting guide 60 to the tibia T. An articulated mechanism 63 may mechanically connect the cutting guide 60 to the robotic base 30. Appropriate joints may be present in the articulated mechanism 63 to allow a movement of the cutting guide 60 relative to the robotic base 30, such as a sliding or telescopic joint 63A, a first rotational joint 63B (e.g., revolute joint), and a second rotational joint 63C (e.g., revolute joint). The joints 63A, 63B and 63C are shown being in a serial arrangement, but other arrangements are considered, such as by combining the joints 63B and 63C in a single rotational joint having two rotational degrees of freedom (e.g., spherical joint, universal joint).

Movements of the cutting guide 60 may be navigated in position and/or orientation through the appropriate electronics 22, 32 that are part of the robotic system 10, so as to provide a desired orientation to the tibial plateau relative to a landmark of the tibia, such as the mechanical axis, the topmost point of the tibial plateau, or deepest point of the tibial plateau. If present, the cameras 32 may optionally be used to provide video imaging by which the cutting guide 60 may be positioned and oriented relative to the bone. A 3D virtual model of the tibial plateau may be used to be overlaid with the footage of the cameras 32 as a reference. Accordingly, in a variant, the positioning of the cutting guide 60 may be based on imaging, for example, with the imaging being used to determine the deepest point on the tibial plateau. Moreover, some or all of the various degrees of freedom in the articulated mechanism 63, between the cutting guide 60 and the bone anchor device 20, may be actuated by the actuators within the robotic tool unit to automate or control the position and/or orientation of the cut slot 61 relative to the tibia T. The bone anchor device 20 that is used in FIGS. 6A to 6C may navigate a mechanical axis of the tibia. Various techniques and tools may be used to calibrate the bone anchor device 20 and enable it to track tibial landmarks, such as those described in U.S. Pat. No. 10,729,452, incorporated herein by reference, according to which the mechanical axis of the tibia T may be digitized and tracked by an inertial sensor, such as the one present in the bone anchor device 20. Thus the orientation of the cut slot 61 may be adjusted in relation to a varus-valgus (e.g., joint 63B) and/or slope (e.g. joint 63C).

Once the cutting guide 60 is appropriately placed relative to the tibia T, the cutting guide 60 may be anchored to the bone, for example by pins in the pinholes 62. Components of the robotic tool unit may be removed, such as the robotic base 30 and the articulated mechanism 63. The bone anchor device 20 may also be removed, or may remain in the tibia T, deep enough so as not to intersect the cut plane of the cut slot 61. If it remains in the tibia T, the bone anchor device 20 may be used for post-operative motion tracking. Moreover, the bone anchor device 20 may be connected to a tibial plateau implant to receive force sensing data from force sensors in the implant.

Referring now to FIGS. 7A to 7C, another approach is shown for creating a proximal tibial plane. The surgical implement of the robotic system 10 includes a milling tool or like cutting tool 70 that is translated onto the bone surface by the articulated mechanism 63. Therefore, as part of FIG. 7A, an orientation of the cutting tool 70 is adjusted to achieve, for example, a desired orientation between the cutting implement 70 and the tibia. Again, various techniques and tools may be used to calibrate the bone anchor device 20 and enable it to track tibial landmarks, such as those described in U.S. Pat. No. 10,874,405, incorporated herein by reference, according to which the mechanical axis of the tibia T may be digitized and tracked by an inertial sensor, such as the one present in the bone anchor device 20. Thus, the orientation of the cutting tool 70 may be adjusted in relation to a varus-valgus (e.g., joint 63B) and/or slope (e.g. joint 63C).The cutting implement 70 may then be translated onto a top surface of the tibial plateau, by way of joint 63A, after having been properly oriented, to resurface the tibial plateau. The robotic system 10 may control the translational movement to achieve a desired resection depth of the tibial plateau. Hence, the articulated mechanism 63 may drive the movement of the cutting implement 70, though manual assistance may be used as well.

The on-bone robotic system 10 featuring the surgical implements 60 and/or 70 may self-align relative to the tibia T, by performing its tibial registration, and may guide tibial cut, or perform the tibial cut itself. If the bone anchor device 20 is an implanted electronic device that is used post-operatively, the coordinates of the plane resulting from the tibial registration may be transferred to the electronic components 22 of the bone anchor device 20, as data used in the post-operative tracking.

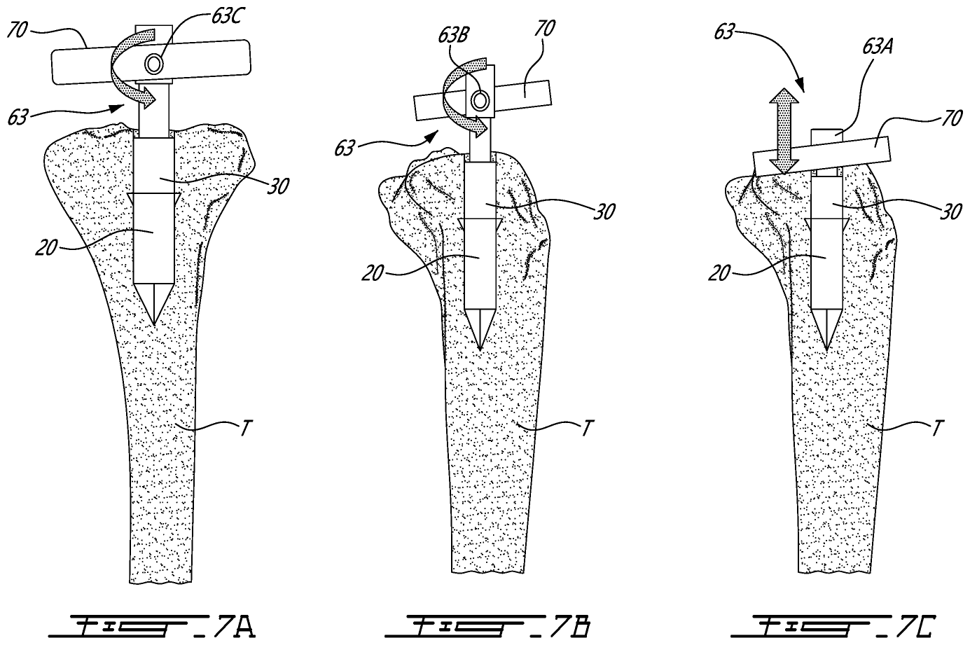

Referring to FIGS. 8A to 8E, the on-bone robotic system 10 is shown using a provisional implant 80, as surgical implement for the robotic tool unit, in conjuction with the robotic base 30, and operating with the bone anchor device 20 described above. The provisional implant surgical implement 80 may be used intraoperatively, after a preliminary cut of the distal femur has been made. The provisional implant surgical implement 80 is used to assist in determining a desired position and/or orientation of the femoral implant relative the femur F, by providing data associated with soft tissue balancing of the bone. The provisional implant surgical implement 80 may therefore have a geometry emulating a shape of a femoral implant, with a distal surface 80A and a posterior surface 80B, the posterior surface 80B having condyle-like formations. The provisional implant surgical implement 80 may have appropriate force sensors, as part of the electronics/mechanical components 42, to gather force data for various flexion-extension and/or varus-valgus angles at the knee. Accordingly, in order to enable soft tissue balancing, the provisional implant surgical implement 80 must be adjustable andmovable relative to the femur F, as described in U.S. Pat. Nos. 7,442,196, 10,555,822, and 10,485,554, which are incorporated by reference herein. Therefore, the preliminary cut(s) made in the distal femur, such as a posterior cut and/or a distal cut, must take into consideration the size of the provisional implant surgical implement 80 to allow movement of the provisional implant surgical implement 80. Moreover, the preliminary cut(s) must be minimal to allow additional bone removal for the final cut(s) to be made for the femoral implant to be installed.

In an embodiment, the provisional implant surgical implement 80 is connected to the robotic base 30 by the spherical joint 33 and/or the translational joint 34 (FIGS. 3A, 3B and 3C), such that the actuators within the on-bone robotic system 10 may lock the provisional implant surgical implement 80 in a given position and orientation, relative to the femur F, the femur F having its landmarks tracked by the bone anchor device 20. The position and/or orientation of the provisional implant surgical implement 80 is tracked relative to the femur F, via the various possible electronic/mechanical components 32,42, such as the encoders, the motors, the linear actuator and/or the inertial sensor. These components may be used in conjunction with the data provided by the inertial sensor in the bone anchor device 20. With the provisional implant surgical implement 80 in a fixed position and orientation relative to the femur, various knee manipulations may be made to gather force sensor data, the force sensor data being correlated to the instant position and orientation of the provisional implant surgical implement 80. Dynamic adjustments may be performed by the on-bone robotic system 10, for instance if the force sensor data is above given thresholds, that may be indicative of soft-tissue unbalance. The dynamic adjustements may be achieved by adjustments to the position and/or orientation of the provisional implant surgical implement 80, such as to reproduce given varus-valgus angles, flexion-extension angles, femur rotation in flexion and/or femur length. Once sufficient data has been acquired by the force sensors of the provisional implant surgical implement 80 to select a target femoral implant position and orientation, the provisional implant surgical implement 80 may be detached. A cutting guide implement, such as that shown at 40 in FIG. 4A or FIG. 4B, may be attached to the robotic base 30, or to the provisional implant surgical implement 80 in another embodiment, to position cut slot(s) at a position and orientation corresponding to the target femoral implant position and orientation, with the geometrical relation and size of the cutting guide implement 40 are taken into consideration.

As part of the surgical workflow involving the provisional implant surgical implement 80, the preliminary cut(s) may be made to the distal femur F to remove sufficient bone for the provisional implant cutting implement 80 to be secured to the femur. The resection of the tibial plateau as shown in FIGS. 6A to 6C and 7A to 7C may be achieved before or after the preliminary cut(s) to the distal femur F. The surgical workflow may thus conclude with resection of the femur to create the appropriate plane cuts, after the soft tissue balancing with the provisional implant surgical implement 80.

Still referring to FIGS. 8A to 8E, an alternative to the use of the joints 33 and 34 is shown, with the distal surface 80A of the provisional implant surgical implement 80 having actuated pads 81A. Likewise, the posterior surface 80B of the provisional implant surgical implement 80 may have actuated pads 81B. Each of the actuated pads 81A, 81B may be displaceable in translation relative to a remainder of the provisional implant surgical implement 80, and may hold set positions relative to the remainder of the provisional implant surgical implement 80. Any appropriate motor or linear actuator from the components 42 may be used to actuate the displacement. The movement to set positions may be used to emulate adjusted position and orientation of the provisional implant surgical implement 80 with respect to the femur F. Therefore, as shown in FIGS. 8A and 8B, the flexion angle may be adjusted. As shown in FIG. 8B, the rotation of the femur in the AP plane may be adjusted for balance. As shown in FIGS. 8C and 8D, the varus-valgus angle may be adjusted. Force sensors as described in U.S. Pat. No. 10,485,554 may be integrated into the actuated pads 81A and/or 81B to measure the forces in dynamic soft tissue balancing maneuvers for various degrees of varus-valgus and flexion-extension. In FIG. 8E, an optional cutting guide implement 82 may be positioned against the actuated pads 81A, via abutment surface 82A, to transfer their combined plane of contact to a cut slot 82B. The cutting guide implement 82 may then be pinned to the bone, and the robotic base 30 may be removed, for the cut plane to be resected. In the embodiments of FIGS. 8A to 8E, the robotic base 30 may be optional, though the robotic base 30 may be used to interface the bone anchor device 20 to the provisional implant surgical implement 80.

The electronic components 42 on board the provisional implant surgical implement 80 may include range finders, such as optical sensors, that may be used to determine distances between the actuated pads 81A and 81B and a remainder of the provisional implant surgical implement 80, or from the provisional implant surgical implement 80 to the bone, to determine position and/or orientation. For example, this may be an alternative to having an inertial sensor. These sensors may be used to determine a distance between the provisional implant surgical implement 80 and the tibial plateau during range of motion and laxity testing. The operator would then be given pressure readings as well as distance readings.

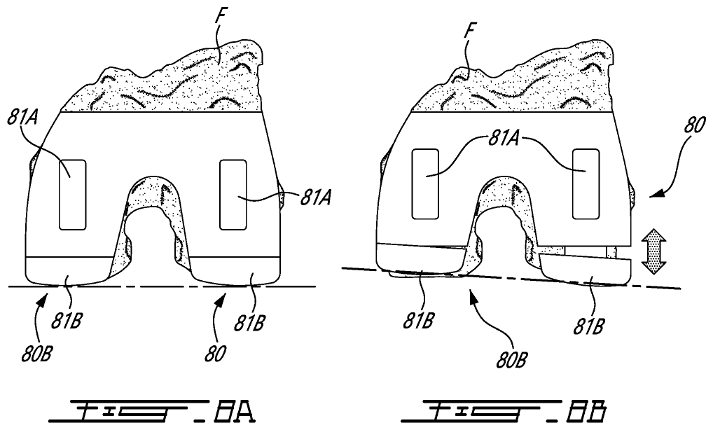

Referring to FIG. 9, another surgical implement is shown at 90. The surgical implement is a cutting block 90 that may be used in various procedures. For instance, the cutting block 90 may be used in machining the distal plane of the femur in the embodiment of FIG. 4, or the tibial plateau in the embodiment of FIGS. 7A-7C, as the cutting block 90 can be used to prepare a planar bone surface.

A housing 91 may include a plurality of cutting heads 92, in a milling tool arrangement, i.e., mill heads. In the example of FIG. 9, the housing 91 is shown having a generally trapezoidal perimeter around the plurality of cutting heads 92. The perimeter can be shaped to complement the shape of a bone surface to be machined (e.g., femur, tibia). Other perimeter shapes can be provided, including generally triangular, parallelogram, rectangular or irregular shapes. The plurality of cutting heads 92 can be disposed within the housing 91 and can be exposable through the attacking surface of the cutting block 90.

The cutting block 90 can be populated with the plurality of cutting heads 92 that are arranged to machine a planar surface. Together, the plurality of cutting heads 92 can form a two-dimensional cutting surface. In some examples, the cutting heads 92 can be extended or retracted with respect to the housing 91 such that the two-dimensional cutting surface can be exposed outside the housing 91. The cutting heads 92 may be operated by motor(s) from the electronic/mechanical components 42. Additional structure may be present oscillate or rotate the cutting heads 92, that may be oscillated or rotated together as a whole. The oscillation or rotation of the cutting heads 92 (e.g., as a whole) can be in addition to rotational or oscillating movement provided to each of the plurality of cutting heads 92. For example, ultrasonic actuation may be used to drive oscillations of the cutting block 90 and/or its displacement toward the bone. Irrigation and suction of bone debris is also planned in the cutting block 90, as shown by suction hole 93A, connected to a suction source S and irrigation jet 93B in order to facilitate the milling operation. Only one suction hole 93A is shown but others could be present, at various locations. Likewise, only one irrigation jet 93B is shown, but others may be present, at various locations.

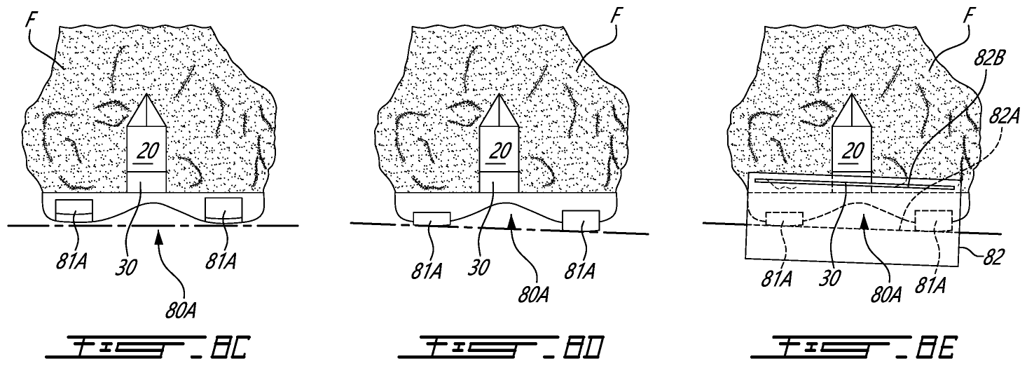

Referring to FIG. 10, another surgical implement is shown at 100. The surgical implement 100 is another cutting block that may be used in various procedures. For instance, the cutting block 100 may also be used in machining the distal plane of the femur in the embodiment of FIG. 4A or FIG. 4B, or the tibial plateau in the embodiment of FIGS. 7A-7C, as the cutting block 100 can be used to prepare a planar bone surface.

The cutting block 100 may include a cutting band 101. The cutting block 100 can also include a first cylindrical drive member 102A and a second cylindrical drive member 102B disposed within housing 103. The cutting band 101 can extend (e.g., be stretched) between the first cylindrical drive member 102A and the second cylindrical drive member 102B. One of the members 102A and 102B may be driven as another possibility. The cutting band 101 can form a closed loop (e.g., a flexible eternal band). The cutting band 101 can be rotated upon activation of a motor from the components 42. In some examples, the rotators can reside inside of the first and/or second cylindrical drive members 102A and/or 102B. In some examples, instead of rotating or in addition to rotating the cutting band, the cutting band can be oscillated upon activation by an oscillator. The cutting band 101 may also be rotated by way of a transmission. Examples of transmissions include tendons and pulleys, chains and sprockets, gear drives, etc. The cutting band 101 can include abrasive elements. In some examples, the abrasive elements are a series of blades. Irrigation and suction of bone debris is also planned in the cutting block 100, as shown by suction hole 104A, connected to a suction source and irrigation jet 104B in order to facilitate the milling operation. Only one suction hole 104A is shown but others could be present, at various locations. Likewise, only one irrigation jet 104B is shown, but others may be present, at various locations.

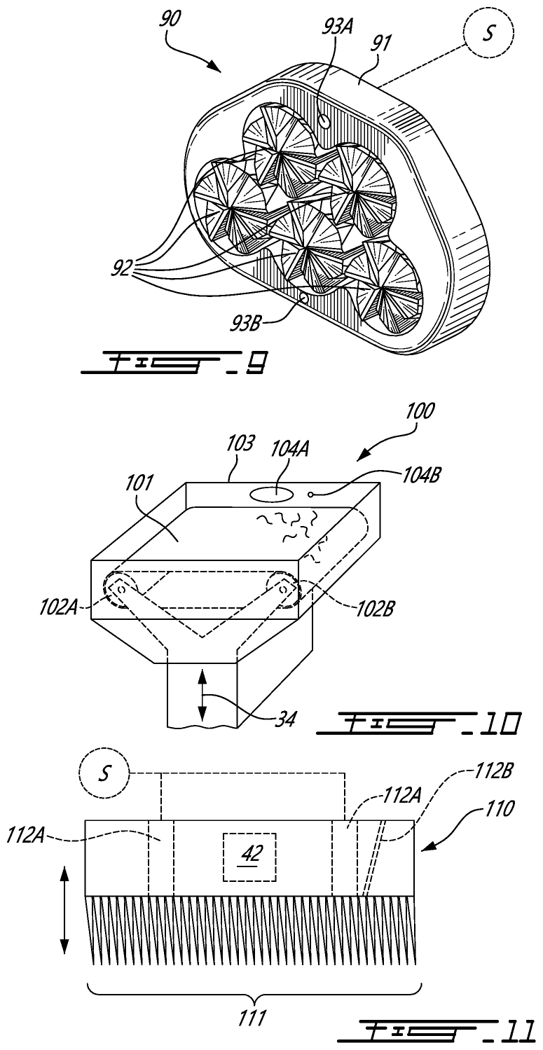

In FIG. 11, another surgical implement is shown at 110. The surgical implement 110 is another cutting block that may be used in various procedures. For instance, the cutting block 110 may also be used in machining the distal plane of the femur in the embodiment of FIG. 4, or the tibial plateau in the embodiment of FIGS. 7A-7C, as the cutting block 110 can be used to prepare a planar bone surface.

The cutting block 110 may feature a plurality of blades 111, that may oscillate when placed against a bone surface, to prepare a planar bone surface. In an embodiment, vertical oscillations of the blades 111, i.e., in an axial direction of the blades 111, are generated to perform a cutting action. Ultrasound actuation may be used to generate the oscillations, i.e., its displacement toward the bone. Irrigation and suction of bone debris is also planned in the cutting block 110, as shown by suction holes 112A, connected to a suction source S and irrigation jet 112B in order to facilitate the milling operation. A pair of suction holes 112A is shown but others could be present (or fewer), at various locations. Likewise, only one irrigation jet 112B is shown, but others may be present, at various locations.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure, and such modifications are intended to fall within the appended claims. While the on-bone robotic system 10 is described as being used for knee surgical, for femur and/or tibia resecton, similar procedure may be used for other bones, such as the the humerus, the spine, etc. For the tibia, an assembly as described in U.S. Pat. No. 10,729,452 may be used, the contents of U.S. Pat. No. 10,729,452 being incorporated herein by reference.

### Claim Related Examples

Example 1 is an on-bone robotic system comprising a bone anchor device configured to be received in a bone, the bone anchor device including at least one sensor for tracking an orientation of the bone; a robotic tool unit releasably connected to the bone anchor device, the robotic tool unit including at least one actuator for displacing a surgical implement of the robotic tool unit relative to the bone when the robotic tool unit is connected to the bone anchor device; wherein the on-bone robotic system includes at least one joint enabling at least one degree of freedom of movement of the surgical implement relative to the bone anchor device; and wherein the on-bone robotic system includes a processor for operating the at least one actuator as a function of the tracking of the bone by the sensor.

Example 2 can include or may optionally be combined with the subject matter of Example 1, wherein the bone anchor device has a receptacle configured to be received in the bone, the receptacle accommodating the at least one sensor.

Example 3 can include or may optionally be combined with the subject matter of Example 2, wherein a leading end of the bone anchor device is flared.

Example 4 can include or may optionally be combined with the subject matter of Examples 2 and 3, wherein an anti-rotation feature projects laterally from the receptacly.

Example 5 can include or may optionally be combined with the subject matter of Example 4, wherein the anti-rotation feature includes at least one fin.

Example 6 can include or may optionally be combined with the subject matter of Examples 1 to 5, wherein the at least one sensor includes an inertial sensor.

Example 7 can include or may optionally be combined with the subject matter of Examples 1 to 6, wherein the bone anchor device includes a battery.

Example 8 can include or may optionally be combined with the subject matter of Example 7, wherein the bone anchor device is configured to be used as an implant to track movement of the bone post-operatively.

Example 9 can include or may optionally be combined with the subject matter of Examples 1 to 8, wherein the at least one actuator includes at least one motor.

Example 10 can include or may optionally be combined with the subject matter of Example 9, including two of the motor, the robotic tool unit displacing the surgical implement in at least two rotational degrees of freedom.

Example 11 can include or may optionally be combined with the subject matter of Examples 1 to 10, wherein the at least one actuator includes at least one linear actuator.

Example 12 can include or may optionally be combined with the subject matter of Examples 1 to 11, wherein the surgical implement has a cut slot.

Example 13 can include or may optionally be combined with the subject matter of Examples 1 to 12, wherein the robotic tool unit includes at least one sensor for tracking an orientation of the surgical implement.

Example 14 can include or may optionally be combined with the subject matter of Examples 1 to 13, wherein the robotic tool unit includes at least one camera oriented toward the bone and configured to capture images of the bone.

Example 15 can include or may optionally be combined with the subject matter of Examples 1 to 14, including a communication device connected to the processor and configured for wireless communication.

Example 16 is a method for performing an orthopedic procedure comprising: anchoring an on-bone robotic system to a bone via a bone anchor device inserted in the bone, the bone anchor device including at least one sensor for tracking an orientation of the bone; operating the on-bone robotic system for the on-bone robotic system to displace a surgical implement operatively connected to the bone anchor device, a movement of the surgical implement being guided as a function of the tracking of the bone by the sensor; and detaching at least the surgical implement from the bone anchor device to leave the bone anchor device as an implant post-operatively, the bone anchor device configured to track the bone post-operatively.

Example 17 can include or may optionally be combined with the subject matter of Example 16, wherein anchoring the on-bone robotic system to the bone including drilling a hole in the bone for insertion of the bone anchor device in the hole.

Example 18 can include or may optionally be combined with the subject matter of Example 17, wherein insertion of the bone anchor device in the hole includes having an anti-rotation feature penetrate the bone.

Example 19 can include or may optionally be combined with the subject matter of Examples 16 to 18, wherein the movement in the operating includes moving the surgical implement in at least one rotational degree of freedom.

Example 20 can include or may optionally be combined with the subject matter of Example 19, wherein moving the surgical implement includes actuating a rotational motor to move the surgical implement in the at least one rotational degree of freedom.

Example 21 can include or may optionally be combined with the subject matter of Examples 19 to 20, wherein the movement in the operating includes moving the surgical implement in two rotational degrees of freedom.

Example 22 can include or may optionally be combined with the subject matter of Examples 19 to 21, wherein the movement in the operating includes moving the surgical implement in one translational degree of freedom.

Example 23 can include or may optionally be combined with the subject matter of Examples 16 to 22, further including imaging the bone from the on-bone robotic system.

Example 24 can include or may optionally be combined with the subject matter of Example 23, further including matching the imaging of the bone from the on-bone robotic system with a pre-operative virtual model of the bone for navigating a position and orientation of the surgical implement relative to the bone.

Example 25 can include or may optionally be combined with the subject matter of Examples 16 to 24, further including wirelessly communicating data from the at least one sensor.

Example 26 is a system for tracking a bone intraoperatively in a surgical procedure and post-operatively, comprising: a processing unit; and a non-transitory computer-readable memory communicatively coupled to the processing unit and comprising computer-readable program instructions executable by the processing unit for: obtaining orientation data of at least one sensor in a bone anchor device anchored to a bone, intraoperatively; actuating at least one actuator to displace a surgical implement operatively connected to the bone anchor device as a part of an on-bone robot, as a function of the orientation data; and after the surgical procedure, obtaining orientation data of at least one sensor in the bone anchor device remaining anchored to the bone, post-operatively.

Example 27 can include or may optionally be combined with the subject matter of Example 26, wherein actuating at least one actuator includes actuating at least one rotational motor to orient the surgical instrument relative to the bone in one rotational degree of freedom.

Example 28 can include or may optionally be combined with the subject matter of Example 26, wherein actuating at least one actuator includes actuating a second rotational motor to orient the surgical instrument relative to the bone in a second rotational degree of freedom.

Example 29 can include or may optionally be combined with the subject matter of Examples 26 to 28, wherein actuating at least one actuator includes actuating at least one linear actuator to displace the surgical instrument relative to the bone in a translational degree of freedom.

Example 30 can include or may optionally be combined with the subject matter of Examples 26 to 29, further including imaging the bone from the on-bone robot.

Example 31 can include or may optionally be combined with the subject matter of Example 30, further including matching the imaging of the bone from the on-bone robot with a pre-operative virtual model of the bone for navigating a position and orientation of the surgical implement relative to the bone.

Each of these non-limiting examples can stand on its own, or can be combined in various permutations or combinations with one or more of the other examples.

1. An on-bone robotic system comprising
a bone anchor device configured to be received in a bone, the bone anchor device including at least one sensor for tracking an orientation of the bone;
a robotic tool unit releasably connected to the bone anchor device, the robotic tool unit including at least one actuator for displacing a surgical implement of the robotic tool unit relative to the bone when the robotic tool unit is connected to the bone anchor device;
wherein the on-bone robotic system includes at least one joint enabling at least one degree of freedom of movement of the surgical implement relative to the bone anchor device; and
wherein the on-bone robotic system includes a processor for operating the at least one actuator as a function of the tracking of the bone by the sensor.

2. The on-bone robotic system according to claim 1, wherein the bone anchor device has a receptacle configured to be received in the bone, the receptacle accommodating the at least one sensor.

3. The on-bone robotic system according to claim 2, wherein a leading end of the bone anchor device is flared.

4. The on-bone robotic system according to claim 2, wherein an anti-rotation feature projects laterally from the receptacly.

5. The on-bone robotic system according to claim 4, wherein the anti-rotation feature includes at least one fin.

6. The on-bone robotic system according to claim 1, wherein the at least one sensor includes an inertial sensor.

7. The on-bone robotic system according to claim 1, wherein the bone anchor device includes a battery.

8. The on-bone robotic system according to claim 7, wherein the bone anchor device is configured to be used as an implant to track movement of the bone post-operatively.

9. The on-bone robotic system according to claim 1, wherein the at least one actuator includes at least one motor.

10. The on-bone robotic system according to claim 9, including two of the motor, the robotic tool unit displacing the surgical implement in at least two rotational degrees of freedom.

11. The on-bone robotic system according to claim 1, wherein the at least one actuator includes at least one linear actuator.

12. The on-bone robotic system according to claim 1, wherein the surgical implement has a cut slot.

13. The on-bone robotic system according to claim 1, wherein the robotic tool unit includes at least one sensor for tracking an orientation of the surgical implement.

14. The on-bone robotic system according to claim 1, wherein the robotic tool unit includes at least one camera oriented toward the bone and configured to capture images of the bone.

15. The on-bone robotic system according to claim 1, including a communication device connected to the processor and configured for wireless communication.

16. A system for tracking a bone intraoperatively in a surgical procedure and post-operatively, comprising:
a processing unit; and
a non-transitory computer-readable memory communicatively coupled to the processing unit and comprising computer-readable program instructions executable by the processing unit for:
obtaining orientation data of at least one sensor in a bone anchor device anchored to a bone, intraoperatively;
actuating at least one actuator to displace a surgical implement operatively connected to the bone anchor device as a part of an on-bone robot, as a function of the orientation data; and
after the surgical procedure, obtaining orientation data of at least one sensor in the bone anchor device remaining anchored to the bone, post-operatively.

17. The system according to claim 16, wherein actuating at least one actuator includes actuating at least one rotational motor to orient the surgical instrument relative to the bone in one rotational degree of freedom.

18. The system according to claim 16, wherein actuating at least one actuator includes actuating a second rotational motor to orient the surgical instrument relative to the bone in a second rotational degree of freedom.

19. The system according to claim 16, wherein actuating at least one actuator includes actuating at least one linear actuator to displace the surgical instrument relative to the bone in a translational degree of freedom.

20. The system according to claim 16, further including imaging the bone from the on-bone robot.
